(19) United States
(12) Patent Application Publication
(10) Pub. No.: US 2017/0266024 A1
(43) Pub. Date: Sep. 21, 2017

(54) STENT

(71) Applicant: TERUMO KABUSHIKI KAISHA, Tokyo (JP)

(72) Inventors: Takashi KUMAZAWA, Fujinomiya-city (JP); Toshihiro YAMAMOTO, Hadano-city (JP)

(73) Assignee: TERUMO KABUSHIKI KAISHA, Tokyo (JP)

(21) Appl. No.: 15/440,617

(22) Filed: Feb. 23, 2017

(30) Foreign Application Priority Data
Mar. 16, 2016 (JP) 2016-053098

(51) Int. Cl. A61F 2/915; A61F 2/04

(52) U.S. Cl. CPC A61F 2/915; A61F 2/04; A61F 2002/91575; A61F 2002/91591; A61F 2210/0004; A61F 2250/0067; A61F 2240/001; A61F 2/07

(57) ABSTRACT

A stent includes a tubular body possessing a plurality of gaps. The tubular body includes a plurality of circumferentially extending linear struts. The stent includes a plurality of links connecting the linear struts. At least one of the links has first and second connection portions. The first connection portion is integrally formed with one strut, and the second connection portion is integrally formed with an adjacent strut. The stent includes a biodegradable material between the first connection portion and the second connection portion to connect the first and second connection portions to each other. The biodegradable material restrains the one strut and the adjacent strut from moving to their original shapes. The first and second connection portions move relative to one another in a separation direction when a connection by the biodegradable material is released so that the original shapes of the struts are restored.

### CROSS-REFERENCES TO RELATED APPLICATIONS

This application claims priority to Japanese Application No. 2016-53098 filed on Mar. 16, 2016, the entire content of which is incorporated herein by reference.

### TECHNICAL FIELD

The present invention generally relates to a stent and a stent manufacturing method.

### BACKGROUND DISCUSSION

A stent needs to possess strength for maintaining an expanded state because the stent is indwelled in a stenosed site or an occlusion site formed inside a body lumen (such as a blood vessel) in the expanded state to maintain an opened state of the body lumen. The stent also needs to have flexibility so that the stent can follow a shape of the body lumen (i.e., generally conform to the surface contour of the body lumen). There have been various attempts for improving flexibility of the stent.

For example, International Patent Application Publication No. 2007/013102 discloses a stent in which struts are connected to each other by a bridge formed of a biodegradable material (a bioabsorbable polymer). Desired flexibility is exhibited when the connection of the struts is released after a predetermined time elapses from the time of the stent being indwelled inside a body lumen.

### SUMMARY

The struts are connected to each other by the bridge while the struts are close to each other. This configuration may lead to the struts still being close to each other even after the connection is released. In this case, there is a possibility that the struts may overlap each other if a force is carelessly (e.g., accidentally) applied to the stent. When the struts overlap each other, the thickness of the stent at the overlapping portion increases and thus an inner diameter of the stent decreases. The possibility of restenosis thus increases because a thrombus or the like more easily occurs in a portion in which the inner diameter decreases in the stent.

The stent disclosed in this application is configured to suppress restenosis after indwelling the stent by preventing struts from overlapping each other.

The stent includes a linear strut which forms a cylindrical outer periphery having gaps formed therein and a plurality of link portions which connect the struts at the gaps. At least one of the link portions includes one connection portion and the other connection portion which are respectively integrally formed with one strut and the other strut adjacent to each other and are disposed to face each other and a biodegradable material which is interposed between the one connection portion and the other connection portion and connects the one connection portion and the other connection portion to each other. The one connection portion and the other connection portion move in a separation direction when a connection by the biodegradable material is released.

In another aspect, the stent includes a tubular body possessing a plurality of gaps. The tubular body includes a plurality of circumferentially extending linear struts. The stent includes a plurality of links connecting the linear struts. At least one of the links has first and second connection portions. The first connection portion is integrally formed with one strut, and the second connection portion is integrally formed with an adjacent strut. The stent includes a biodegradable material between the first connection portion and the second connection portion to connect the first and second connection portions to each other. The biodegradable material restrains the one strut and the adjacent strut from moving to their original shapes. The first and second connection portions move relative to one another in a separation direction when a connection by the biodegradable material is released so that the original shapes of the struts are restored.

Another stent disclosed in this application includes a tubular body extending in an axial direction and possessing a circumferential direction. The tubular body is insertable into a living body. The tubular body includes a plurality of linear struts extending in the circumferential direction. The linear struts are spaced apart from one another with gaps between adjacent linear struts. Each of the linear struts includes a connection portion. The stent includes a link having biodegradable material. The link connects the connection portion of a first strut to the connection portion of a second strut adjacent to the first strut. The biodegradable material degrades over a time period within the living body to release the connection. The connection portion of the first strut is close to the connection portion of the second strut in both the axial and circumferential directions. The first and second struts each possess an original shape. The biodegradable material of the link restrains the first strut from moving to the original shape of the first strut and restrains the second strut from moving to the original shape of the second strut before the time period elapses and the biodegradable material degrades. The first and second struts move to separate when the biodegradable material degrades and releases the connection of the connection portion of the first strut to the connection portion of the second strut so that the first strut is restored to the original shape of the first strut and the second strut is restored to the original shape of the second strut.

According to the stent with the above-described configuration, the connection portions connecting one strut and the other strut adjacent to each other are adapted to move in the separation direction when the connection of the link portion is released. This configuration makes it possible to prevent the connection portions from overlapping each other after the connection of the link portion is released. As a result, it is possible to suppress restenosis caused by a thrombus or the like because an unexpected decrease in inner diameter of the stent is prevented.

In another aspect, the disclosure here relates to a stent manufacturing method that includes applying a restraining force to move a first connection portion of a first linear strut from a first original position and to move a second connection portion of a second linear strut from a second original position. The first and second linear struts extend in a circumferential direction. The first and second linear struts do not overlap one another in the circumferential direction when the first connection portion is in the first original position and the second connection portion is in the second original position. The restraining force moves the first connection portion of the first linear strut and the second connection portion of the second linear strut in the circumferential direction to a restrained position in which the first connection portion and the second connection portion are close to one another. The method includes fixing the first connection portion of the first strut and the second connection portion of the second strut relative to one another while the restraining force is being applied to hold the first connection portion and the second connection portion in the restrained position in which the first and second connection portions are close to one another. The fixing is accomplished using a biodegradable material

### BRIEF DESCRIPTION OF THE DRAWINGS

FIG. 1 is a perspective view of a stent of an embodiment.

FIG. 2 is a development view in which a part of an outer periphery of the stent of the embodiment is cut linearly along the axial direction.

FIG. 3A is an enlarged view of an embodiment of the link portion of the stent, and FIG. 3B is an enlarged cross-sectional view taken along a line 3B-3B of FIG. 3A.

FIG. 4A is a partially enlarged view of the stent before a connection of an embodiment of the link portion is released, and FIG. 4B is a partially enlarged view of the stent after the connection of an embodiment of the link portion is released.

FIG. 5 is a diagram provided to illustrate an arrangement of a connection portion in an embodiment of the link portion.

FIG. 6 is a diagram provided to describe an arrangement of the connection portion after the connection of an embodiment of the link portion is released.

FIG. 7 is a flowchart illustrating a stent manufacturing method of the stent embodiment shown in FIG. 1.

FIG. 8A is a schematic diagram illustrating an embodiment of a stent manufacturing apparatus, and FIG. 8B is an enlarged view showing a molding die.

FIG. 9 is an enlarged view of a part B surrounded by a two-dotted chain line of FIG. 8B and illustrates part of the stent manufacturing method.

FIG. 10 is an enlarged view of the part B surrounded by the two-dotted chain line of FIG. 8B and illustrates another part of the stent manufacturing method.

FIG. 11A is an enlarged view of a link portion of a stent of a modified example, and FIG. 11B is an enlarged cross-sectional view taken along a line 11B-11B of FIG. 11A.

FIG. 12 is a diagram that illustrates an arrangement of a connection portion in a link portion of a modified example.

FIG. 13 is a diagram that illustrates an arrangement of the connection portion after a connection of the link portion of the modified example of FIG. 12 is released.

### DETAILED DESCRIPTION

Set forth below with reference to the accompanying drawings is a detailed description of embodiments of a stent and a method for manufacturing the stent representing examples of the inventive stent and stent manufacturing method disclosed here. The dimension ratios in the drawings may be exaggerated for convenience of description and different from the real dimension ratios.

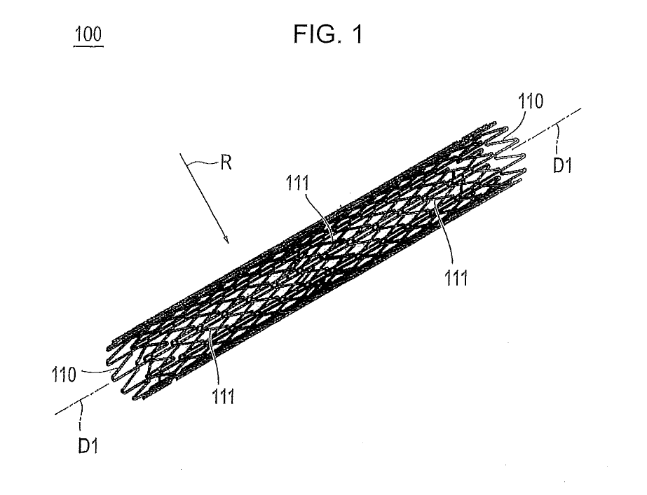

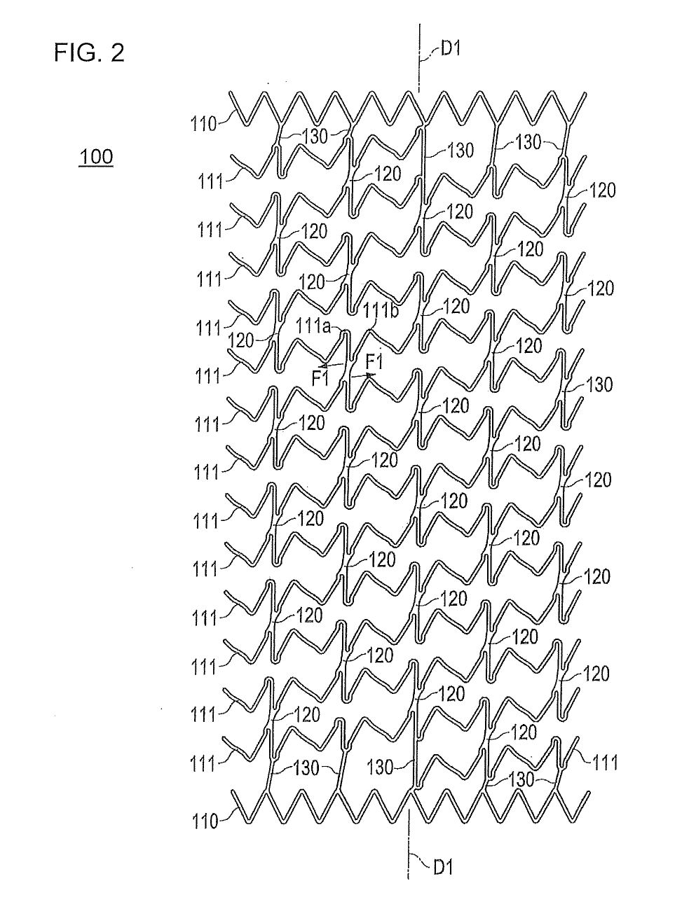

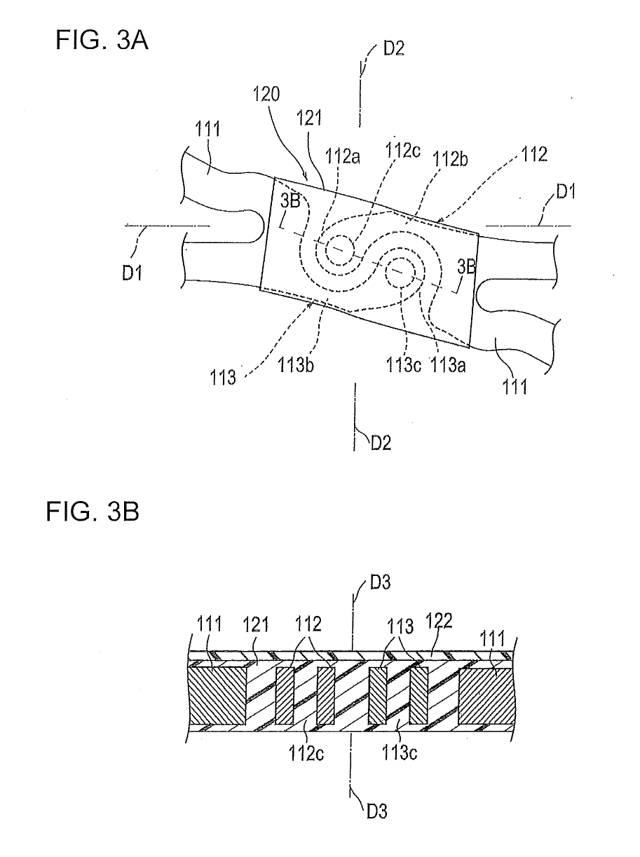

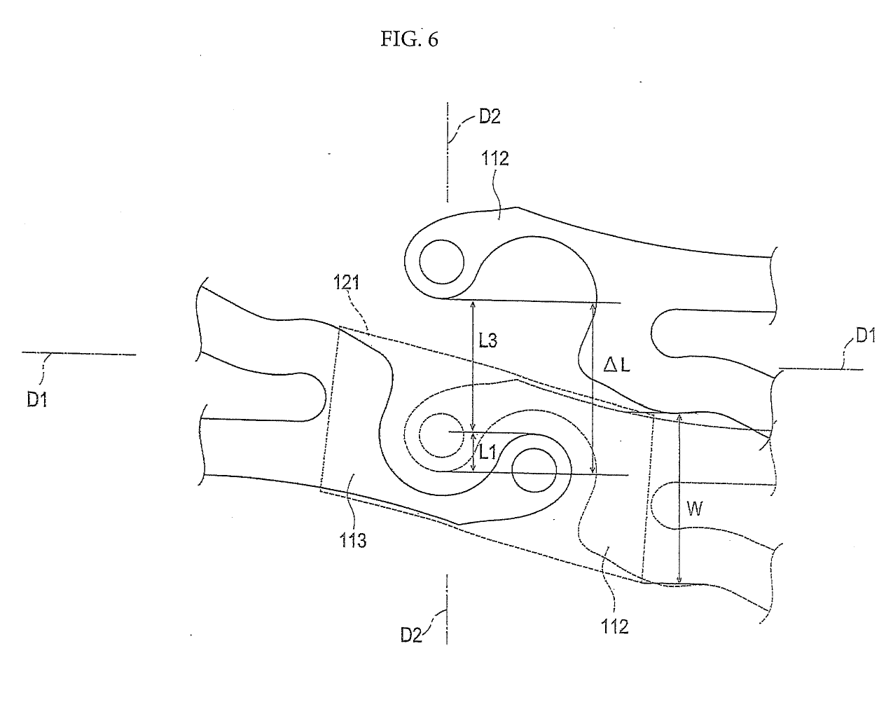

FIGS. 1 and 2 are schematic diagrams showing a structure of an embodiment of a stent 100. FIGS. 3A to 5 are schematic diagrams showing the structure of a link portion 120 of the stent 100 illustrated in FIGS. 1 and 2. FIG. 6 is a diagram illustrating a movement of the stent 100 shown in FIGS. 1 and 2. A connection portion 112 and a biodegradable material 121 before a connection of the link portion 120 is released are indicated by a dotted line in FIG. 6. One embodiment of the stent 100 is described below with reference to FIGS. 1 to 6.

As shown in FIGS. 1 and 2, the stent 100 includes struts 110 and 111 which are linear elements (i.e., elements configured to extend linearly). The stent 100 also includes a plurality of link portions 120 and 130. The struts 110 and 111 form a cylindrical outer periphery having gaps formed in the cylindrical outer periphery.

The axial direction of the cylindrical shape which is formed by the struts 110 and 111 will be referred to in this specification as the “axial direction D1” (see FIG. 1), the circumferential direction of the cylindrical shape will be referred to as the “circumferential direction D2” (see FIG. 3A), the thickness direction of the cylindrical shape will be referred to as the “thickness direction D3” (see FIG. 3B), and the radial direction of the cylindrical shape will be referred to as the “radial direction R” (see FIG. 1).

The strut 110 is located at both ends in the axial direction D1 and extends in the circumferential direction D2 to form an endless annular shape (i.e., a hollow ring shape).

The strut 111 extends in a helical shape about the axial direction D1 between the strut 110 at one end and the strut 110 at the other end. The strut 111 includes a plurality of apexes 111a and 111b which are bent while being turned back in a waved shape.

The material forming the struts 110 and 111 is, for example, a non-biodegradable material which is not biodegraded in a living body.

The material forming the strut 111 is deformable by an external force and restorable into an original shape when a binding action caused by the external force is released. For example, the strut 111 material may be an elastic material including stainless steel, cobalt alloy such as cobalt-chromium alloy (for example, CoCrWNi alloy), elastic metal such as platinum-chromium alloy (for example, PtFeCrNi alloy), and super-elastic alloy such as nickel-titanium alloy. The restoring force (i.e., the force to return the strut 111 to the original shape) represents an elastic force of an elastic material.

The strut 110 material is not particularly limited, but can be the same material as the strut 111.

The link portion 120 connects a strut 111 (e.g., a first strut) and an adjacent strut 111 (e.g., a second strut), which are adjacent to each other at a gap formed between the strut 111 and the adjacent strut 111.

The link portions 120 are positioned in a direction along the axial direction D1.

As shown in FIG. 3A, the link portion 120 includes the first connection portion 112, the second connection portion 113, and the biodegradable material 121. The first connection portion 112 and the second connection portion 113 will generally be referred to in this description as the “connection portions 112 and 113.”

The connection portions 112 and 113 are respectively integrally formed with the strut 111 and the adjacent strut 111 (i.e., two struts 111 axially adjacent to each other) and are connected to each other by the biodegradable material 121 while facing each other.

The first connection portion 112 is formed such that a part of one strut 111 of the two adjacent struts 111 partially protrudes, and the second connection portion 113 is formed such that a part of the other strut 111 partially protrudes. In other words, the first connection portion 112 is a protruding part of one strut 111, and the second connection portion 113 is a protruding part of the adjacent strut 111.

As shown in FIGS. 3A and 3B, the first connection portion 112 includes a protruding portion 112a. The protruding portion 112a protrudes toward the second connection portion 113 and has a curved and rounded shape. The first connection portion 112 also includes a housing portion 112b which is continuous to the protruding portion 112a (i.e., integrally formed with the protruding portion 112a) and has a concave shape in response to an outer shape of a protruding portion 113a of the second connection portion 113 (i.e., the concave shape of the housing portion 112b is positioned directly opposite the convex shape of the protruding portion 113a to face the protruding portion 113a as illustrated in FIG. 3A). The first connection portion 112 includes a holding portion 112c which is formed to penetrate the strut 111 in the thickness direction D3 and contains the biodegradable material 121. The second connection portion 113 includes a protruding portion 113a which protrudes toward the first connection portion 112 and has a curved and rounded shape, a housing portion 113b which is continuous to (i.e., integrally formed with) the protruding portion 113a and has a concave shape in response to an outer shape of the protruding portion 112a of the first connection portion 112 (i.e., the concave shape of the housing portion 113b is positioned directly opposite the convex shape of the protruding portion 112a as illustrated in FIG. 3A), and a holding portion 113c which is formed to penetrate the strut 111 in the thickness direction D3 and contains the biodegradable material 121.

The concave shape of the housing portion 112b is larger (longer) than the outer shape of the protruding portion 113a. The concave shape of the housing portion 113b is also larger (longer) than the outer shape of the protruding portion 112a.

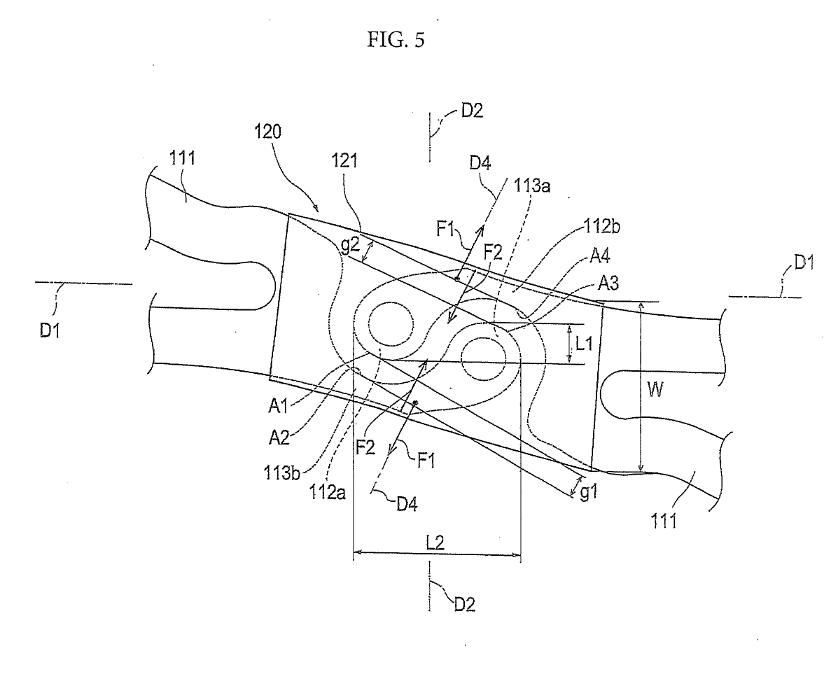

As shown in FIG. 5, the protruding portion 112a is positioned (housed) in the concave shape of the housing portion 113b. A gap g1 is formed between a face A1 (an outer surface of the protruding portion 112a) which faces the housing portion 113b in the protruding portion 112a and a face A2 (an outer surface of the housing portion 113b) which faces the protruding portion 112a in the housing portion 113b when the protruding portion 112a is positioned in the housing portion 113b as illustrated in FIG. 5.

The protruding portion 113a is positioned (housed) in the concave shape of the housing portion 112b. A second gap g2 is formed between a face A3 (an outer surface of the protruding portion 113a) which faces the housing portion 112b in the protruding portion 113a and a face A4 (an outer surface of the housing portion 112b) which faces the protruding portion 113a in the housing portion 112b when the protruding portion 113a is positioned in the housing portion 112b.

In some embodiments, the protruding portion 112a may partially contact the housing portion 113b. The protruding portion 113a may also partially contact the housing portion 112b in some embodiments.

The connection portions 112 and 113 are close to one another in both the circumferential direction D2 and the axial direction D1. The connection portions 112 and 113 are positioned to overlap each other on a virtual line parallel in the axial direction D1 and on a virtual line parallel in the circumferential direction D2 while being connected to each other by the biodegradable material 121. The length of an overlapping portion in the circumferential direction D2 on the virtual line parallel in the axial direction D1 of the connection portions 112 and 113 is indicated by distance L1 of FIG. 5. The length of an overlapping portion in the axial direction D1 on the virtual line parallel in the circumferential direction D2 of the connection portions 112 and 113 is indicated by distance L2 of FIG. 5.

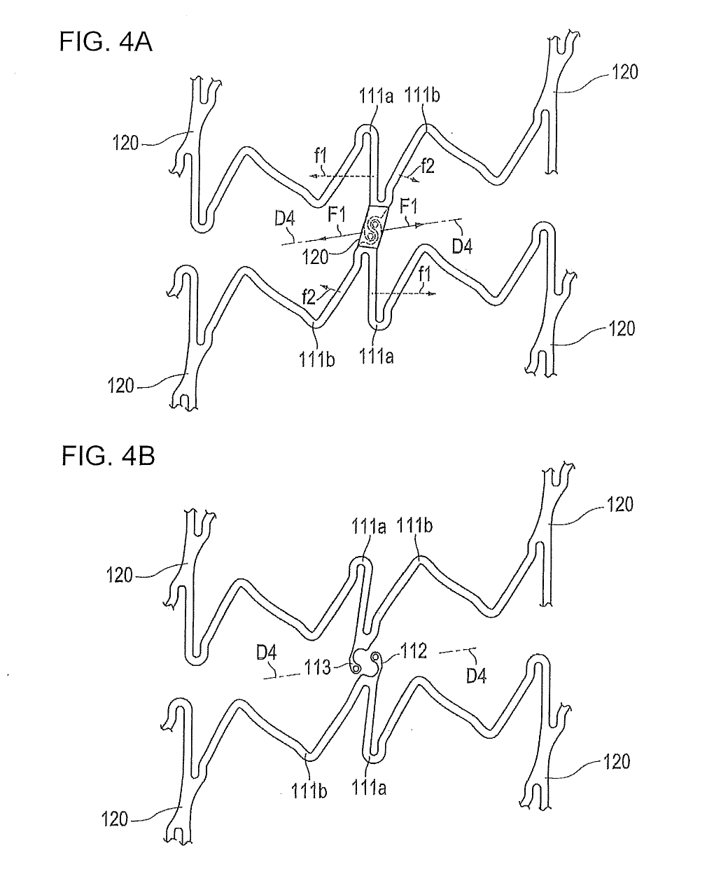

As shown in FIG. 4A, the apexes 111a and 111b are connected to each other by the link portion 120 while being elastically deformed in a direction in which the connection portions 112 and 113 move close to each other. The apexes 111a and 111b respectively have restoring forces f1 and f2 which are forces urging the apexes 111a and 111b to be restored to the original shapes of the apexes 111a and 111b. When the restoring force f1 and the restoring force f2 are added to each other (i.e., resulting in a combined force), a force F1 acting on the connection portions 112 and 113 is obtained.

The restoring force f1 of the apex 111a is larger than the restoring force f2 of the apex 111b by using a manufacturing process described below. Accordingly, the force F1 acting on the connection portions 112 and 113 is exerted in a direction D4 in which the connection portions 112 and 113 are separated from each other as shown in FIG. 5. When the biodegradable material 121 biodegrades to thereby release the connection, the connection portions 112 and 113 move in the separation direction D4 because the force F1 acts on the connection portions 112 and 113 as shown in FIG. 4B.

The force F1 acting on the connection portions 112 and 113 due to the restoring forces f1 and f2 of the struts 111 will be referred to as the “separating force F1.”

The “separation direction D4” is formed so that the gaps g1 and g2 (see FIG. 5) respectively formed between the faces A1 and A3 of the protruding portions 112a and 113a and the faces A2 and A4 of the housing portions 112b and 113b are widened. In other words, when the connection portions 112 and 113 separate based on the separating force F1, the distance between the outer surface of the protruding portion 112a and the outer surface of the housing portion 113b increases, and similarly the distance between the outer surface of the protruding portion 113a and the outer surface of the housing portion 112b increases.

As shown in the embodiment of FIG. 3B, each of the holding portions 112c and 113c is formed as a penetration hole penetrating the strut 111 in the thickness direction D3. Each of the holding portions 112c and 113c does not need to be a penetration hole (i.e., a through-hole) as long as the biodegradable material 121 can be contained in the holding portions 112c and 113c. The holding portions 112c and 113c may be formed in a shape which is recessed to a certain degree in the thickness direction D3 of at least the strut 111.

As shown in FIGS. 3A and 3B, the biodegradable material 121 ties the first connection portion 112 and the second connection portion 113 to each other (i.e., holds or fixes the first connection portion 112 and the second connection portion 113 to one another) until the biodegradable material is biodegraded after a predetermined time elapses from the time of indwelling the stent 100 in a body lumen. As shown in FIG. 5, the biodegradable material 121 maintains a state where a restricting force F2 acts on the connection portions 112 and 113 against the separating force F1 of the connection portions 112 and 113. The movement of the connection portions 112 and 113 in the separation direction D4 is thus limited by the restricting force F2.

The biodegradable material 121 is provided to be integrally connected to the surfaces of the connection portions 112 and 113, the gap between the first connection portion 112 and the second connection portion 113, and the inside of each of the holding portions 112c and 113c. Since the biodegradable material 121 covers the surfaces of the connection portions 112 and 113, fills the gap between the first connection portion 112 and the second connection portion 113, and fills the inside of each of the holding portions 112c and 113c, it is possible to satisfactorily tie (hold or fix) the connection portions 112 and 113 to each other.

The biodegradable material 121 is not particularly limited as long as the material biodegrades in a living body. Examples of the biodegradable material 121 include a biodegradable synthetic polymer such as polylactic acid, polyglycolic acid, lactic acid-glycolic acid copolymer, polycaprolactone, lactic acid-caprolactone copolymer, glycolic acid-caprolactone copolymer, and poly-γ-glutamic acid, a biodegradable natural polymer such as collagen, or a biodegradable metal such as magnesium and zinc.

As shown in FIG. 3B, the stent 100 includes a cover member 122 that includes a drug and is formed on the surface of the stent 100. The cover member 122 is desirably formed on an outer surface of the stent 100 facing an inner peripheral face of the body lumen, but the stent disclosed in this application is not limited to this configuration.

The cover member 122 includes a drug configured to suppress (capable of suppressing) a growth of a neo-intima and a drug loading member loading the drug. In another embodiment, the cover member 122 may be formed only by the drug. The drug included in the cover member 122, for example, is at least one of a group including sirolimus, everolimus, zotarolimus, paclitaxel, and the like. A material forming the drug loading member is not particularly limited. However, a biodegradable material is desirably used, and the same material as that of the biodegradable material 121 can be employed.

The link portion 130 is integrally formed with the strut 110 and the strut 111 as shown in FIG. 2.

Next, an example of a method of manufacturing the stent 100 will be described.

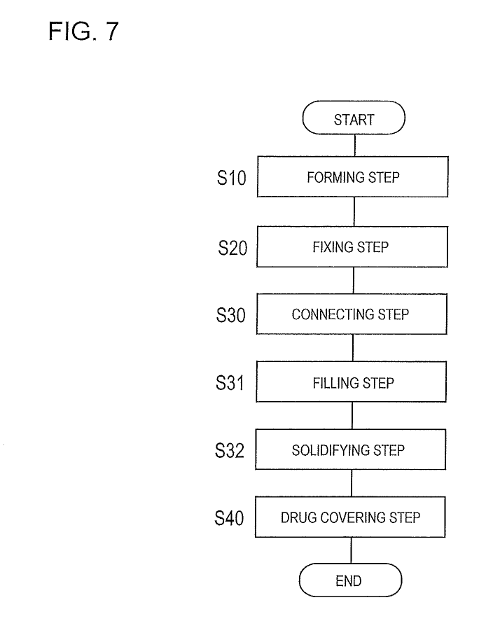

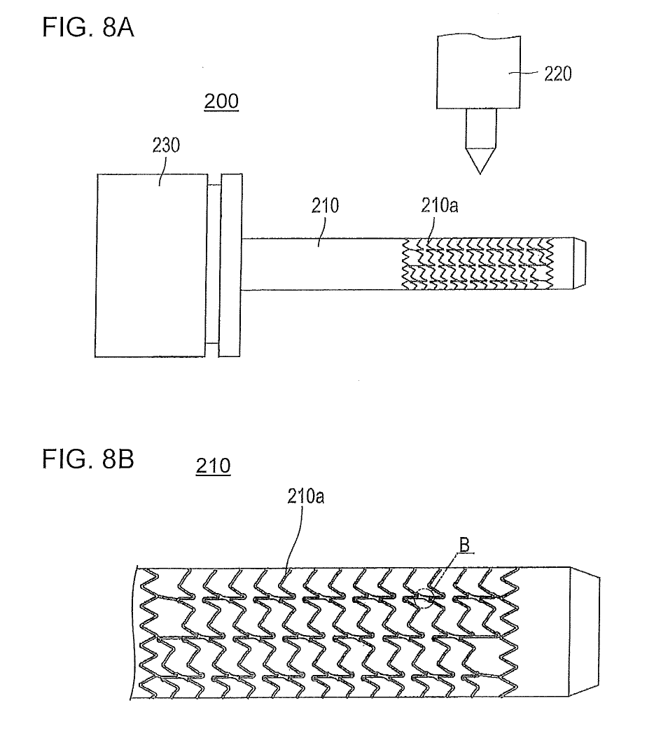

FIG. 7 is a flowchart illustrating an example of a method of manufacturing the stent 100. FIGS. 8 to 10 are schematic diagrams showing an example of a manufacturing apparatus 200 configured to manufacture the stent 100.

The manufacturing apparatus 200 used to manufacture the stent 100 is not particularly limited as long as the method of manufacturing the stent 100 shown in FIG. 7 can be performed. For example, the manufacturing apparatus 200 may include a columnar molding die 210, a filling device 220 which fills the biodegradable material 121, and a support member 230 that supports the molding die 210 as shown in FIG. 8A. The support member 230 supports the molding die 210 so that the molding die 210 is rotatable in the circumferential direction of the molding die 210 and is movable in the axial direction of the molding die 210. A groove 210a which corresponds to a shape of the stent 100 is formed at an outer surface of the molding die 210 as shown in FIG. 8B.

A method of manufacturing the stent 100 (a stent manufacturing method) is illustrated in FIG. 7 and includes a forming step (S10) of forming a stent body 10, a fixing step (S20) of fixing the stent body 10 to the molding die 210, a connecting step (S30) of connecting the connection portions 112 and 113 to each other by the biodegradable material 121, and a drug covering step (S40).

In the forming step (S10), a portion corresponding to a gap of the stent 100 is removed from a metallic tube (which is a stent material). The stent body 10 is thus formed. The stent body 10 includes an annular body formed by the strut 110, the strut 111 which extends in a helical shape about the axial direction D1, and the link portion 130 which integrates the strut 110 and the strut 111 with each other is formed. The stent body 10 possesses a cylindrical shape with a gap.

A portion corresponding to the gap of the stent 100 is appropriately removed by an etching method called photo-fabrication and by using masking and chemicals, a discharge machining method using a die, a cutting method, or the like. The cutting method is, for example, mechanical polishing or laser cutting. Finishing such as chemical polishing or electrolytic polishing or heat treatment such as annealing is subsequently appropriately performed.

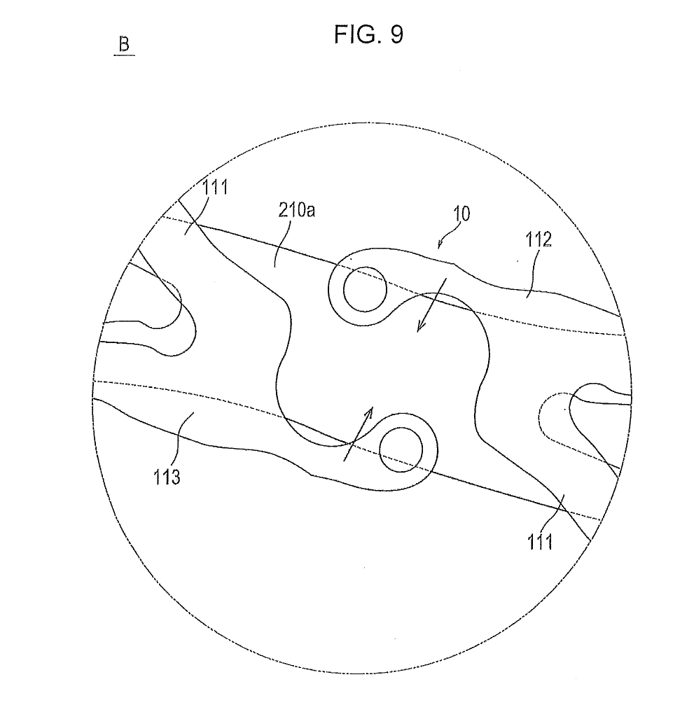

The stent body 10 is positioned in the groove 210a of the molding die 210 to be fixed thereto in the fixing step (20). The stent body 10 is disposed on the outer surface of the molding die 210, and the molding die 210 is inserted through the stent body 10. At this time, the apexes 111a and 111b of the strut 111 are bent to be elastically deformed by an external force applied in a direction indicated by an arrow of FIG. 9 so that the connection portions 112 and 113 move closer to each other while exhibiting reaction forces against the restoring forces f1 and f2 of the strut 111 (see FIG. 4A). Subsequently, the connection portions 112 and 113 are inserted and fixed into the groove 210a while maintaining the reaction forces.

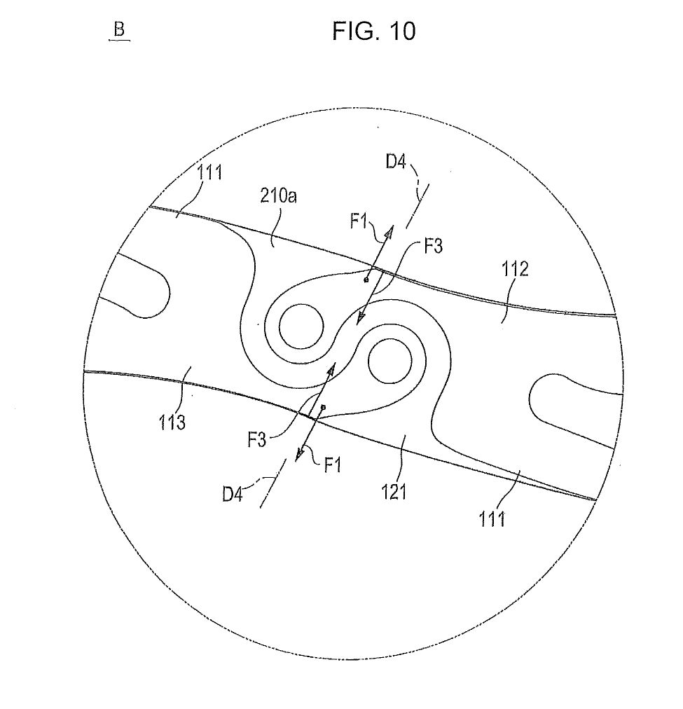

The separating force F1 acts on the connection portions 112 and 113 due to the restoring forces f1 and f2 of the struts 111 as shown in FIG. 10. In this state, the connection portions 112 and 113 receive a reaction force F3 acting against the separating force F1 from an inner face of the groove 210a (i.e., the surface of the groove 210a). For this reason, the struts 111 and the connection portions 112 and 113 are strongly fixed (held in place) by the groove 210a. Since the groove 210a of the molding die 210 is used to fix the connection portions 112 and 113, it is possible to mold the stent 100 with high accuracy by suppressing a deviation in arrangement of the connection portions 112 and 113 of the link portion 120.

In the connecting step (S30), the connection portions 112 and 113 (which are fixed to the molding die 210) are connected to each other by the biodegradable material 121 to form the link portion 120. The connecting step (S30) includes a filling step (S31) of filling the biodegradable material 121 into the groove 210a of the molding die 210 and a solidifying step (S32) of solidifying the biodegradable material 121 that has filled the groove 210a of the molding die 210.

In the filling step (S31), for example, a liquid droplet of the biodegradable material 121 is ejected into the groove 210a by a filling device 220 such as a micro syringe so that the biodegradable material 121 is interposed between the first connection portion 112 and the second connection portion 113 (see FIG. 10). The ejected biodegradable material 121 intrudes into the gap between the first connection portion 112 and the second connection portion 113 and each of the holding portions 112c and 113c by a capillary phenomenon. Accordingly, it is possible to fill the biodegradable material 121 into the groove 210a of the molding die 210.

The biodegradable material 121 can be continuously filled into the groove 210a of the outer surface of the molding die 210. For example, the molding die 210 supported by the support member 230 can be rotated in the circumferential direction or moved in the axial direction by a driving device such as a motor when the biodegradable material 121 is filled into the groove 210a.

A polymer solution obtained by dissolving the biodegradable material 121 in a solvent can be filled into the groove 210a of the molding die 210 when the biodegradable material 121 is a polymer, such as a biodegradable synthetic polymer or a biodegradable natural polymer. The solvent material, for example, can be an organic solvent such as methanol, ethanol, dioxane, tetrahydrofuran, dimethylformamide, acetonitrile, dimethylsulfoxide, and acetone.

A liquid biodegradable metal which is melted by heat may be filled (injected) into the groove 210a of the molding die 210 by the filling device 220 when the biodegradable material 121 is a biodegradable metal.

Since the amount of the biodegradable material 121 forming the link portion 120 is defined by the volume of the groove 210a of the molding die 210, a quantitative amount of the biodegradable material 121 can be filled. In other words, a predetermined amount of biodegradable material 121 can be injected into the groove 210a to fill the groove 210a. The degradation speed of the biodegradable material 121 of each link portion 120 can be thus be uniform because each link portion 120 can be formed by a predetermined amount of biodegradable material 121. The release of the connection of the link portion 120 can thus be stably controlled.

In the solidifying step (S32), the filled biodegradable material 121 solidifies to form the link portion 120. The link portion 120 connects the connection portions 112 and 113 to each other by the biodegradable material 121.

When a polymer solution including a biodegradable synthetic polymer or a biodegradable natural polymer is used to fill the gap 210a, the polymer solution can be solidified in such a way that the polymer solution is dried to evaporate the solvent. A method of drying the polymer solution is, for example, natural drying, but the invention is not limited to natural drying. The polymer solution may be dried by heating. The polymer solution is solidified by drying to form the link portion 120. In other embodiments, the biodegradable material 121 may be melted in such a way that the polymer solution is dried and is further heated. The biodegradable material can be intruded (applied) between the first connection portion 112 and the second connection portion 113 because the biodegradable material 121 fluidity increases due to the melting.

When the biodegradable material 121 is a biodegradable metal, the filled liquid biodegradable metal is cooled to be solidified. A method of cooling the biodegradable material 121 is, for example, air cooling, but the invention is not limited to air cooling. A forced cooling using a cooling device or the like may be employed.

In the drug covering step (S40), the cover member 122 including a drug is formed on an outer surface of the stent 100 facing the inner peripheral face of the body lumen as shown in FIG. 3B.

First, both the drug and the drug loading member are dissolved in a solvent to form a coating solution. The solvent is, for example, acetone, ethanol, chloroform, or tetrahydrofuran.

Next, the coating solution is coated on the surface of the biodegradable material 121 and is dried to evaporate the solvent so that the stent 100 is formed by a drug and a polymer.

Finally, the stent 100 manufacture is completed by being removed from the molding die 210.

Next, an operation and an effect of the stent 100 of the embodiment will be described.

The stent 100 is delivered to, for example, a stenosed site or an occlusion site formed inside a body lumen (such as a blood vessel, a bile duct, a tracheal, an esophagus, or a urethra) by the use of a stent delivery system, such as a balloon catheter. The delivered stent 100 is indwelled in a lesion site such as the stenosed site of the body lumen in an expanded state (i.e., the stent 100 indwells in the body lumen when the stent is in the expanded state).

The biodegradable material 121 in one embodiment of the stent 100 slowly biodegrades at an acute stage which has a possibility of a retreatment and in which a slight (i.e., a relatively small amount) time elapses from the indwelling operation. The connection of the link portion 120 is satisfactorily maintained by the connection portions 112 and 113 as shown in FIG. 3A. For this reason, for example, a device such as a catheter for an IVUS (Intravascular Ultrasound) or an OFDI (Optical Frequency Domain Imaging) used to check an indwelled state or a balloon catheter for a post-expansion operation easily passes through the stent 100 because the stent 100 has a relatively high strength and reliably maintains a largely expanded state immediately after the indwelling operation.

Since the stent 100 maintains a high strength, it is possible to suppress the risk of the stent 100 being deformed in the axial direction D1 even when the above-described example devices unexpectedly contact the stent 100 when passing through the stent 100.

When a stage enters a chronic stage after endothelialization, the link portion 120 releases the connection by the biodegradation of the biodegradable material 121. The stent is easily deformed in the circumferential direction D2 to follow a shape of a curved or meandered body lumen because the stent 100 has improved flexibility.

When the connection of the link portion 120 is released, the restriction imparted by the biodegradable material 121 is released so that the restricting force F2, which is applied to the connection portions 112 and 113 and against the separating force F1, disappears (see FIG. 5). The connection portions 112 and 113 accordingly move in the separation direction D4 to positions not overlapping each other in the axial direction D1 by the separating force F1 as illustrated in FIG. 6.

The distance in the circumferential direction D2 that the first connection portion 112 moves relative to the second connection portion 113 when the connection by the biodegradable material 121 is released is indicated by ΔL. A maximal (maximum) width in the circumferential direction D2 of the link portion 120 before the connection by the biodegradable material 121 is released is indicated by W (see FIG. 5). In the embodiment illustrated in FIG. 6, a relationship between ΔL and W satisfies ΔL≧W (i.e., the first connection portion 112 a distance equal to or greater than the width of the link portion 120 before the connection is released). FIG. 6 shows the movement of the first connection portion 112 relative to the second connection portion 113 for convenience of description.

The “maximal width W in the circumferential direction D2 of the link portion 120” is the largest distance in the circumferential direction D2 between two arbitrary points at a portion provided with the biodegradable material 121 in the top view of the link portion 120 (i.e., a top view in the thickness direction D3) as shown in FIG. 5.

Here, ΔL indicates the relationship between the shortest separation distance L3 in the circumferential direction D2 between the first connection portion 112 and the second connection portion 113 after the first connection portion 112 moves and the length L1 in the circumferential direction D2 at an overlapping portion on a virtual line parallel in the axial direction D1 between the first connection portion 112 and the second connection portion 113 before the first connection portion 112 moves and satisfies ΔL=L1+L3.

The stent 100 has particularly high flexibility and flexibly follows a shape of the body lumen as a result of the connection by the biodegradable material 121 at the link portion 120 being released. It is thus possible to maintain the stent 100 in an opened state while supporting the body lumen in a minimally invasive state for a long period of time.

As described above regarding one embodiment of the stent 100, the connection portions 112 and 113 of the stent 100 move in the separation direction D4 when the connection by the biodegradable material 121 is released. This relative movement between the connection portions 112 and 113 makes is possible to prevent the connection portions 112 and 113 from overlapping each other after the biodegradable material 121 is biodegraded so that the connection between the connection portions 112 and 113 is released. This configuration makes it possible to suppress restenosis caused by a thrombus by preventing an unexpected decrease in inner diameter of the stent 100 after the stent 100 is indwelled (e.g., the connection portions 112 and 113 are prevented from overlapping one another).

The connection portions 112 and 113 are connected to each other by the biodegradable material 121 while the separating force F1 is exerted in the separation direction D4. When the biodegradable material 121 is biodegraded, the connection portions 112 and 113 are released from being restricted by the biodegradable material 121. The connection portions 112 and 113 thus move in the separation direction D4 due to the separating force F1. Accordingly, it is possible to prevent the connection portions 112 and 113 from overlapping each other after the connection between the connection portions 112 and 113 is released.

The connection portions 112 and 113 are disposed at positions overlapping each other on a virtual line parallel in the axial direction D1 while being connected to each other by the biodegradable material 121. This position makes it possible to satisfactorily maintain the connection between the connection portions 112 and 113 (i.e., maintain a connection state). When the connection by the biodegradable material 121 is released, the connection portions 112 and 113 move to positions not overlapping each other on the virtual line parallel in the axial direction D1 (i.e., the connection portions 112 and 113 move relative to one another so that the connections portions 112 and 113 do not overlap in the circumferential direction). Since the connection portions 112 and 113 are disposed at the positions not overlapping each other on the virtual line parallel in the axial direction D1 even when the stent 100 deforms in the axial direction D1 after the connection by the biodegradable material 121 is released, it is possible to further reliably prevent the connection portions 112 and 113 from overlapping each other.

The length ΔL in the circumferential direction D2 is the distance by which the first connection portion 112 moves relative to the second connection portion 113 when the connection by the biodegradable material 121 is released. The length ΔL is equal to or larger than the maximal (maximum) width W in the circumferential direction D2 of the link portion 120. Since the connection portions 112 and 113 further reliably move to the positions not overlapping each other on the virtual line parallel in the axial direction D1, it is possible to further reliably prevent the connection portions 112 and 113 from overlapping each other.

When the connection portions are connected to each other by the biodegradable material 121, the protruding portion 112a is housed in the housing portion 113b and the protruding portion 113a is housed in the housing portion 112b. The protruding portions 112a and 113a at one side and the housing portions 112b and 113b at the other side are disposed at positions overlapping each other on a virtual line parallel in the circumferential direction D2. In other words, the protruding portion 112a and the housing portion 113b are at the same position in the axial direction, and the protruding portion 113a and the housing portion 112b are at the same position in the axial direction (i.e., the connection portions 112 and 113 overlap one another on a virtual line parallel to the axial direction). For this reason, it is possible to satisfactorily maintain the connection between the connection portions 112 and 113.

The strut 111 is formed of an elastic material. The apex 111a of the strut 111 is thus elastically deformed so that the connection portions 112 and 113 move in the separation direction D4 when the connection by the biodegradable material 121 is released. That is, since the strut 111 is formed of an elastic material, the strut can be restored to the original shape of the strut 111 even when the stent 100 is deformed due to a force carelessly (e.g., accidentally) applied from the circumferential direction D2 after the connection by the biodegradable material 121 is released. Since it is possible to stably separate the connection portions 112 and 113 from each other after the connection by the biodegradable material 121 is released, it is possible to further reliably prevent the connection portions 112 and 113 from overlapping each other.

The link portion 120 is provided with the cover member 122. A drug configured to suppress a growth of a neo-intima is gradually eluted from the cover member 122 so that it is possible to further suppress restenosis of a lesion site.

### Modified Example

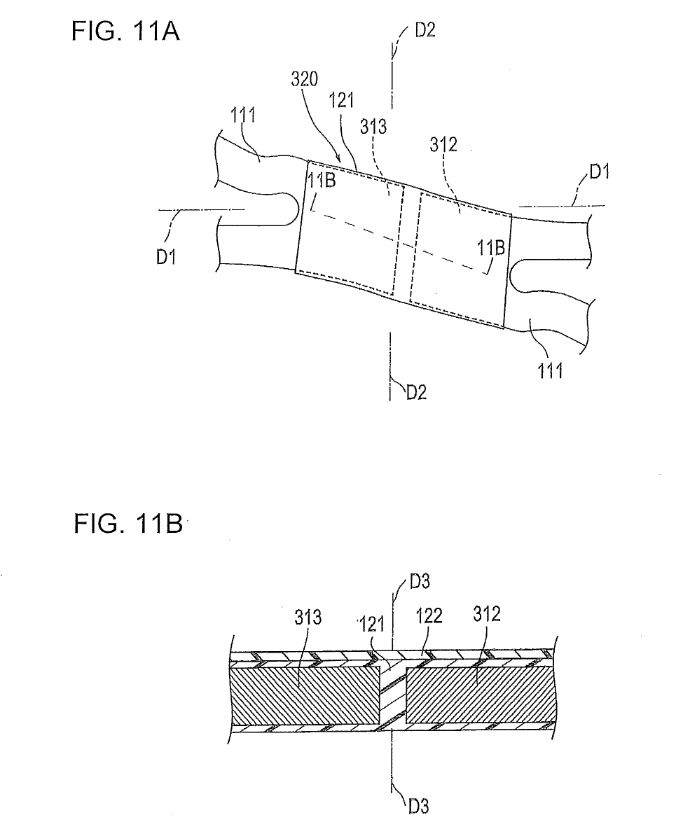

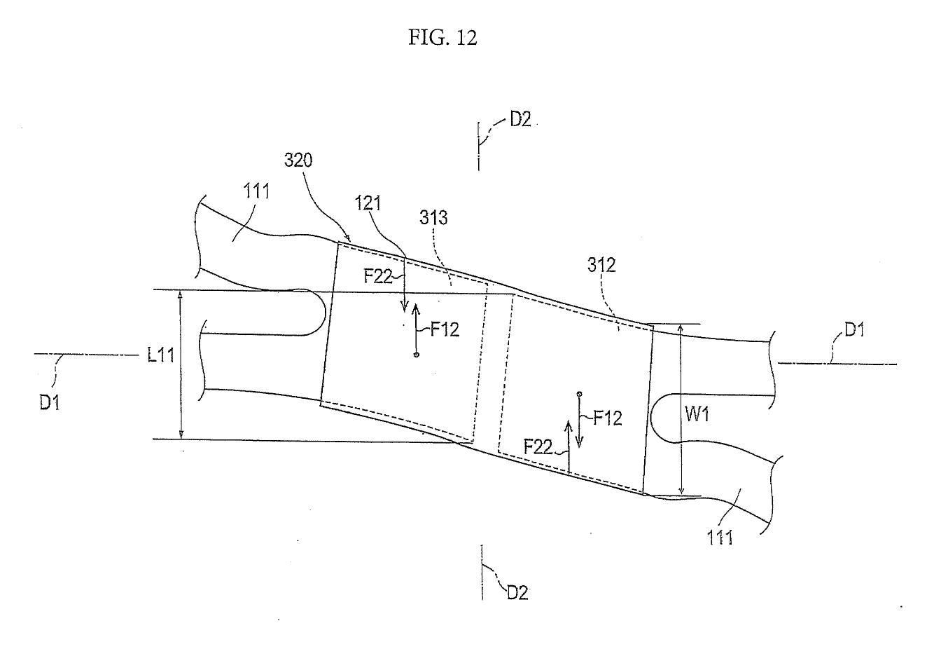

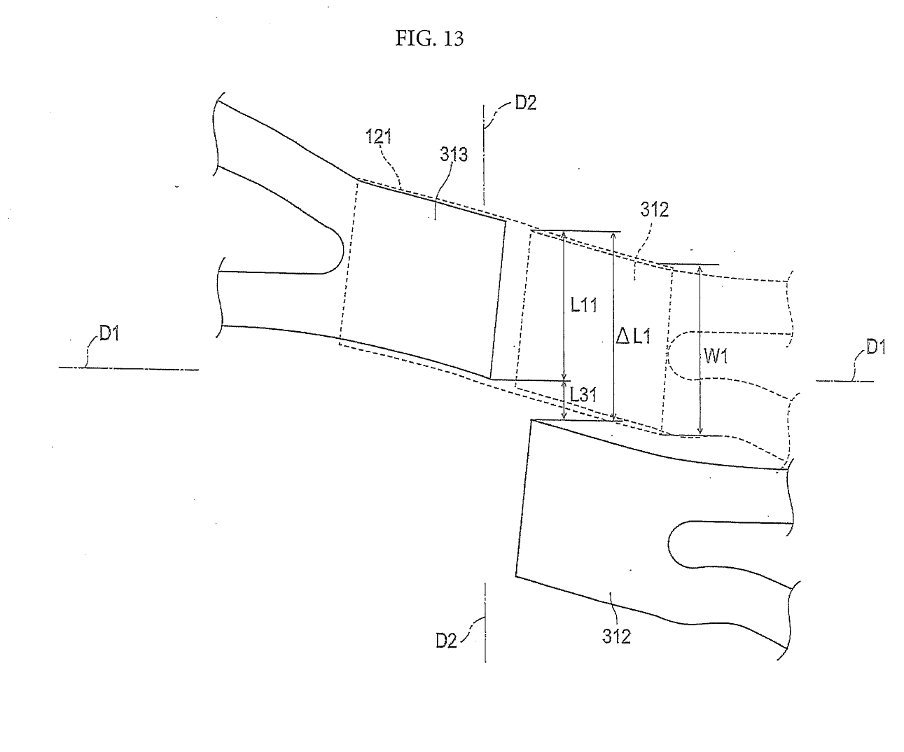

FIGS. 11 and 12 are schematic diagrams showing a structure of a modification example of a link portion 320. FIG. 13 is a diagram provided to illustrate an arrangement of connection portions 312 and 313 after a connection of the link portion 320 is released. In FIG. 13, the first connection portion 312 and the biodegradable material 121 before the connection of the link portion 320 is released are indicated by a dotted line. The same reference numerals will be given to the same configuration elements as those of the embodiment described above and a description of the previously discussed elements will be omitted.

The link portion 320 of a stent 300 according to the modified example is illustrated in FIG. 11. The first connection portion 312 and the second connection portion 313 are connected to each other by the biodegradable material 121. The first connection portion 312 and the second connection portion 313 are close to one another in the circumferential direction. The first connection portion 312 and the second connection portion 313 face each other at positions overlapping each other on a virtual line parallel in the axial direction D1, but are disposed at positions not overlapping each other on a virtual line parallel in the circumferential direction D2 (in contrast to the link portion 120 of the embodiment described above). Hereinafter, the link portion 320 of the modified example will be described.

The first connection portion 312 and the second connection portion 313 are respectively integrally formed with the strut 111 and the adjacent strut 111 which are connected to each other by the biodegradable material 121 to face each other. The first connection portion 312 is disposed at the gap of the second connection portion 313.

The first connection portion 312 is formed such that a part of one strut 111 of two adjacent struts 111 extends toward the other strut 111 to have a rectangular shape and the second connection portion 313 is formed such that a part of the other strut 111 extends toward one strut 111 to have a rectangular shape.

The connection portions 312 and 313 are positioned to overlap each other on a virtual line parallel in the axial direction D1 (i.e., the connection portions 312 and 313 are close to one another in the circumferential direction) while being connected to each other by the biodegradable material 121. A length in the circumferential direction D2 at an overlapping portion on the virtual line parallel in the axial direction D1 between the connection portions 312 and 313 is indicated by L11.

Similarly to the embodiment described above, when the connection portions are connected to each other by the biodegradable material 121, a separating force F12 is applied to the first connection portion 312 and the second connection portion 313 in the separation direction by the restoring force of the strut 111 (i.e., the restoring force urges the strut 111 to return to the original shape). The biodegradable material 121 applies a restricting force F22 against the separating force F12 to the connection portions 312 and 313 to limit the movement of the connection portions 312 and 313 in the separation direction.

The “separation direction” is the circumferential direction D2 of the stent 300. Since the separation direction is the circumferential direction D2, it is possible to suppress a deformation amount of the stent 300 in the axial direction D1.

When the biodegradable material 121 biodegrades over a time period so that the connection between the connection portions 312 and 313 is released as illustrated in FIG. 13, the connection portions 312 and 313 become independent from the restriction by the biodegradable material 121. The connection portions 312 and 313 thus move in the circumferential direction D2 corresponding to the separation direction by the separating force F12. Accordingly, it is possible to reliably prevent the connection portions 312 and 313 from overlapping each other.

The distance that the first connection portion 312 moves relative to the second connection portion 313 in the circumferential direction D2 when the connection by the biodegradable material 121 is released is indicated by ΔL1 in FIG. 13. The maximum width in the circumferential direction D2 of the link portion 320 is indicated by W1 (see FIG. 12). Similarly to the embodiment described above, the relationship between ΔL1 and W1 satisfies ΔL1≧W1. FIG. 13 also shows the movement of the first connection portion 312 relative to the second connection portion 313 for convenience of description.

Here, ΔL1 indicates a relationship between the shortest separation distance L31 in the circumferential direction D2 between the first connection portion 312 and the second connection portion 313 after the first connection portion 312 moves (i.e., the strut returns to the original shape) and the length L11 in the circumferential direction D2 at an overlapping portion on a virtual line parallel in the axial direction D1 between the first connection portion 312 and the second connection portion 313 before the first connection portion 312 moves (i.e., the strut is in the restrained position). The ΔL1 relationship satisfies the equation ΔL1=L11+L31.

In the link portion 320 according to the modified example, the connection portions 312 and 313 move in the circumferential direction D2 when the connection by the biodegradable material 121 is released. The connection portions 312 and 313 can thus move to positions not overlapping each other on the virtual line parallel in the axial direction D1 (i.e., move to not overlap in the circumferential direction) by a minimal movement. Accordingly, it is possible to suppress restenosis by further reliably preventing the connection portions 312 and 313 from overlapping each other.

The invention is not limited to the embodiment and the modified example described above and can be modified into various forms within the scope of claims.

For example, the separating forces F1 and F12 acting on the connection portions 112, 113, 312, and 313 of the link portions 120 and 320 while the connection portions are connected by the biodegradable material 121 are generated by the restoring forces f1 and f2 of the materials forming the apexes 111a and 111b of the struts 111. The configuration in which the separating force acts on the connection portion, however, is not limited to these illustrative separating forces. For example, a configuration may be employed in which the opposite connection portions are formed of a material having a magnetic force pushing the connection portions away from each other and the separating force is generated by the magnetic force. A configuration may also be employed in which the strut is formed of a thermally deformable material such as thermoplastic resin or shape memory alloy and the separating force is generated by a thermal deformation of the strut. The shape of the strut is not particularly limited as long as the separating force is generated.

The apexes 111a and 111b of the strut 111 may be formed of a material having a restoring force and the entire strut 111 does not need to be formed of a material having a restoring force.

A method of manufacturing the stents 100 and 300 is not limited to the embodiments and the modified examples described above and can be appropriately modified in response to the configuration of the link portions 120 and 320 or the struts 110 and 111.

The type of the link portion is not limited to the embodiments and the modified examples described above as long as at least one link portion includes the first connection portion, the second connection portion, and the biodegradable material. For example, in the embodiment described above, the link portion 130 may be formed by the first connection portions 112 and 312, the second connection portions 113 and 313, and the biodegradable material 121 similarly to the link portion 120.

The arrangement of the link portion is also not limited to the embodiments and the modified examples described above and can be appropriately changed.

The embodiment of the strut is not also limited to the embodiments and the modified examples described above. For example, the stent may not include a strut which is similar to the strut 111 described above which extends in a helical shape about the axial direction D1, but may include a strut which is similar to the strut 110 of the embodiment described above and extends in the circumferential direction D2 about the axial direction D1 while being turned back in a waved shape to thereby form an endless annular shape.

The outer shapes of the protruding portion, the housing portion, and the holding portion are not limited to the embodiments and the modified examples described above. For example, the outer shapes of the protruding portion, the housing portion, and the holding portion can be formed in arbitrary polygonal shapes.

The struts 110 and 111 of the embodiment described above are formed of a non-biodegradable material, but the stent disclosed here is not limited to having non-biodegradable struts. The struts may be formed of a biodegradable material biodegrades slower than the biodegradable material included in the link portion.

There is another embodiment of the stent of this application that may not include the cover member 122 and an embodiment including a drug configured to suppress a growth of a neo-intima and provided in the biodegradable material 121. In the latter embodiment, the drug is gradually eluted in accordance with the biodegradation of the biodegradable material 121 and thus restenosis of a lesion site is suppressed.

The detailed description above describes a stent and a stent manufacturing method. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A stent comprising:
a tubular body possessing a plurality of gaps, the tubular body extending in an axial direction and possessing a circumferential direction;
the tubular body comprising a plurality of linear struts extending in the circumferential direction, the gaps being between the linear struts;
a plurality of links connecting the linear struts at the gaps;
at least one of the links comprising a first connection portion and a second connection portion, the first connection portion being integrally formed with one strut of the linear struts and the second connection portion being integrally formed with an other strut of the linear struts adjacent to the one strut and positioned to face the one strut, the one strut and the other strut each possessing an original shape;
a biodegradable material between the first connection portion and the second connection portion to connect the first connection portion and the second connection portion to each other, the biodegradable material restraining the one strut from moving to the original shape of the one strut and restraining the other strut from moving to the original shape of the other strut; and
the first connection portion and the second connection portion moving relative to one another in a separation direction when a connection by the biodegradable material is released so that the one strut is restored to the original shape of the one strut and the other strut is restored to the original shape of the other strut.

2. The stent according to claim 1, wherein the one strut and the other strut exert a force on the first connection portion and the second connection portion in the separation direction when the first connection portion and the second connection portion are connected to each other by the biodegradable material.

3. The stent according to claim 1, wherein
the first connection portion and the second connection portion are close to each other in the circumferential direction of the tubular body while being connected to each other by the biodegradable material, and
when the connection by the biodegradable material is released, the first connection portion and the second connection portion move to positions not overlapping each other in the circumferential direction of the tubular body.

4. The stent according to claim 1, wherein the first connection portion moves a distance in the circumferential direction relative to the second connection portion when the connection by the biodegradable material is released, the distance being equal to or larger than a maximum width of each of the links in the circumferential direction.

5. The stent according to claim 1, wherein
the first connection portion includes a protruding portion and a housing portion extending from the protruding portion, the protruding portion protruding toward the second connection portion, the housing portion possessing a concave shape,
the second connection portion includes a protruding portion and a housing portion extending from the protruding portion, the protruding portion protruding toward the first connection portion, the housing portion possessing a concave shape,
when the connection portions are connected to each other by the biodegradable material, the protruding portion of the first connection portion is positioned in the housing portion of the second connection portion to face the housing portion of the second connection portion, the protruding portion of the first connection portion being close to the housing portion of the second connection portion, and
when the connection portions are connected to each other by the biodegradable material, the protruding portion of the second connection portion is positioned in the housing portion of the first connection portion to face the housing portion of the first connection portion, the protruding portion of the second connection portion being close to the housing portion of the first connection portion.

6. The stent according to claim 1, wherein
the one strut is an elastic material, and
when the connection by the biodegradable material is released, the one strut elastically deforms so that the first connection portion and the second connection portion move in the separation direction.

7. The stent according to claim 1, wherein the separation direction is the circumferential direction of the tubular body.

8. The stent according to claim 1, comprising a cover member provided on an outer surface of the tubular body, the cover member comprising a drug configured to suppress a growth of a neo-intima.

9. A stent comprising:
a tubular body extending in an axial direction and possessing a circumferential direction, the tubular body being insertable into a living body;
the tubular body comprising a plurality of linear struts extending in the circumferential direction, the linear struts being spaced apart from one another with gaps between adjacent linear struts, each of the linear struts comprising a connection portion;
a link comprising biodegradable material, the link connecting the connection portion of a first strut of the linear struts to the connection portion of a second strut of the linear struts adjacent to the first strut, the biodegradable material degrading over a time period within the living body to release the connection of the connection portion of the first strut to the connection portion of the second strut;
the connection portion of the first strut being close to the connection portion of the second strut in both the axial direction and the circumferential direction of the tubular body;
the first strut and the second strut each possessing an original shape;
the biodegradable material of the link restraining the first strut from moving to the original shape of the first strut and restraining the second strut from moving to the original shape of the second strut before the time period elapses and the biodegradable material degrades; and
the first strut and the second strut moving to separate when the biodegradable material degrades and releases the connection of the connection portion of the first strut to the connection portion of the second strut so that the first strut is restored to the original shape of the first strut and the second strut is restored to the original shape of the second strut.

10. The stent according to claim 9, wherein the first strut comprises a plurality of apexes, the connection portion of the first strut extending from one of the apexes of the first strut.

11. The stent according to claim 9, wherein the connection portion of the first strut comprises a first convex portion and a first concave portion, and the connection portion of the second strut comprises a second convex portion and a second concave portion.

12. The stent according to claim 11, wherein
the first convex portion of the first strut is close to the second concave portion of the second strut in the axial direction of the tubular body when the link connects the connection portion of the first strut to the connection portion of the second strut, and
the second convex portion of the second strut is close to the first concave portion of the first strut in the axial direction of the tubular body when the link connects the connection portion of the first strut to the connection portion of the second strut.

13. The stent according to claim 11, wherein
the first convex portion of the first strut is close to the second concave portion of the second strut in the circumferential direction of the tubular body when the link connects the connection portion of the first strut to the connection portion of the second strut,
the second convex portion of the second strut is close to the first concave portion of the first strut in the circumferential direction of the tubular body when the link connects the connection portion of the first strut to the connection portion of the second strut,
the first convex portion of the first strut does not overlap the second concave portion of the second strut in the circumferential direction of the tubular body when the biodegradable material degrades to release the connection of the connection portion of the first strut to the connection portion of the second strut, and
the second convex portion of the second strut does not overlap the first concave portion of the first strut in the circumferential direction of the tubular body when the biodegradable material degrades to release the connection of the connection portion of the first strut to the connection portion of the second strut

14. The stent according to claim 9, wherein the connection portion of the first strut and the connection portion of the second strut each comprises a through hole, the biodegradable material filling each of the through holes when the link connects the connection portion of the first strut to the connection portion of the second strut.

15. A stent manufacturing method comprising:
applying a restraining force to move a first connection portion of a first linear strut from a first original position and to move a second connection portion of a second linear strut from a second original position, the first linear strut and the second linear strut extending in a circumferential direction, the first linear strut and the second linear strut not overlapping one another in the circumferential direction when the first connection portion is in the first original position and the second connection portion is in the second original position;
the restraining force moving the first connection portion of the first linear strut and the second connection portion of the second linear strut in the circumferential direction to a restrained position in which the first connection portion and the second connection portion are close to one another;
fixing the first connection portion of the first strut and the second connection portion of the second strut relative to one another while the restraining force is being applied to hold the first connection portion and the second connection portion in the restrained position in which the first and second connection portions are close to one another; and
the fixing being accomplished using a biodegradable material.

16. The stent manufacturing method according to claim 15, wherein the applying of the restraining force comprises positioning the first connection portion of the first strut and the second connection portion of the second strut in a groove.

17. The stent manufacturing method according to claim 16, wherein the applying of the restraining force further comprises positioning the first strut and the second strut in the groove on an outer surface of a molding die so that the first strut and the second strut extend circumferentially around the outer surface of the molding die.

18. The stent manufacturing method according to claim 16, further comprising introducing the biodegradable material into the groove by a filling device.

19. The stent manufacturing method according to claim 18, further comprising coating an outer surface of both the first strut and the second strut with a drug.

20. The stent manufacturing method according to claim 19, wherein the coating with the drug is performed after the fixing of the first connection portion of the first strut and the second connection portion of the second strut.
